(19) United States
(12) Patent Application Publication
(10) Pub. No.: US 2025/0246309 A1
(43) Pub. Date: Jul. 31, 2025

(54) INFORMATION PROCESSING DEVICE, INFORMATION PROCESSING SYSTEM, AND IDENTIFICATION METHOD

(71) Applicant: Mitsubishi Electric Corporation, Tokyo (JP)

(72) Inventors: Wataru SASAKI, Tokyo (JP); Tatsuhiko ITOHARA, Tokyo (JP)

(73) Assignee: Mitsubishi Electric Corporation, Tokyo (JP)

(21) Appl. No.: 18/854,101

(22) PCT Filed: Apr. 27, 2022
(86) PCT No.: PCT/JP2022/019023

§ 371 Date: Oct. 4, 2024

(51) Int. Cl. G16H 50/30; G16H 20/70

(52) U.S. Cl. CPC G16H 50/30; G16H 20/70

(57) ABSTRACT

An information processing device includes an acquisition unit that acquires a stressor action identification table indicating a correspondence relationship between personality information and action information, personality information indicating personality of a first user, stress information as information regarding stress on the first user, and action information indicating an action of a second user and an identification control unit that judges whether a relationship between the personality information and the action information has been registered in the stressor action identification table or not based on the stress information if a stress value is greater than or equal to a predetermined threshold value and identifies the action information as a stressor action if the relationship has been registered in the stressor action identification table.

### TECHNICAL FIELD

The present disclosure relates to an information processing device, an information processing system, an identification method and an identification program.

### BACKGROUND ART

A person feels stress in daily life. For example, a person feels interpersonal stress. Here, a technology for inferring whether the stress is interpersonal stress or not has been proposed (see Patent Reference 1). For example, a stress management system in the Patent Reference 1 infers whether the stress is interpersonal stress or not by using a user's biological data and life-log data indicating action history records. Further, the stress management system infers the type of the interpersonal stress by using the life-log data.

### PRIOR ART REFERENCE

### Patent Reference

Patent Reference 1: Japanese Patent Application Publication No. 2018-037073

### NON-PATENT REFERENCE

Non-patent Reference 1: Oliver P. John, Laura P. Naumann, and Christopher J. Soto, “Paradigm Shift to the Integrative Big Five Trait Taxonomy”, Handbook of Personality: Theory and Research 3.2 (2008), pp. 114-158
Non-patent Reference 2: June P. Tangney, Roy F. Baumeister, and Angie Luzio Boone, “High Self-Control Predicts Good Adjustment, Less Pathology, Better Grades, and Interpersonal Success”, Journal of Personality 72.2 (2004), pp. 271-324
Non-patent Reference 3: Wu Youyou, Michal Kosinski, and David Stillwell, “Computer-based Personality Judgments are More Accurate than Those Made by Humans”, Proceedings of the National Academy of Sciences 112.4 (2015), pp. 1036-1040
Non-patent Reference 4: Clemens Stachl et al., “Predicting Personality from Patterns of Behavior Collected with Smartphones”, Proceedings of the National Academy of Sciences 117.30 (2020), pp. 17680-17687

### SUMMARY OF THE INVENTION

### Problem to be Solved by the Invention

In the above-described technology, the type of the interpersonal stress is inferred. Here, the type of the interpersonal stress is referred to as a stressor action. In short, the stressor action is an interpersonal action being a factor of stress.

In the above-described technology, the stressor action is inferred by using the life-log data. However, in the method using the life-log data, there are cases where identification accuracy of the stressor action is low.

An object of the present disclosure is to increase the identification accuracy of the stressor action.

### Means for Solving the Problem

An information processing device according to an aspect of the present disclosure is provided. The information processing device includes an acquisition unit that acquires stressor action identification information indicating a correspondence relationship between personality information and action information, personality information indicating personality of a first user, stress information as information regarding stress on the first user, and action information indicating an action of a second user and an identification control unit that judges whether a relationship between the personality information and the action information has been registered in the stressor action identification information or not based on the stress information and identifies the action information as a stressor action if the relationship has been registered in the stressor action identification information.

### Effect of the Invention

According to the present disclosure, the identification accuracy of the stressor action can be increased.

### BRIEF DESCRIPTION OF THE DRAWINGS

FIG. 1 is a diagram showing an information processing system in a first embodiment.

FIG. 2 is a diagram showing hardware included in an information processing device in the first embodiment.

FIG. 3 is a diagram showing hardware included in a terminal in the first embodiment.

FIG. 4 is a sequence diagram showing an example of a process executed between the information processing device and a personality inference device in the first embodiment.

FIG. 5 is a sequence diagram showing an example of a process executed between the information processing device and a stress information generation device in the first embodiment.

FIG. 6 is a sequence diagram showing an example of a process executed between the information processing device and an action inference device in the first embodiment.

FIG. 7 is a block diagram showing functions of the information processing device in the first embodiment.

FIG. 8 is a diagram showing an example of a personality information management table in the first embodiment.

FIG. 9 is a diagram showing an example of a stress information management table in the first embodiment.

FIG. 10 is a diagram showing an example of an action information management table in the first embodiment.

FIG. 11 is a diagram showing an example of a stressor action identification table in the first embodiment.

FIG. 12 is a diagram showing an example of an instrument proposal table in the first embodiment.

FIG. 13 is a flowchart showing an example of a process executed by the information processing device in the first embodiment.

FIG. 14 is a flowchart showing an example of a stressor action identification process in the first embodiment.

FIG. 15 is a flowchart showing an example of an instrument proposal process in the first embodiment.

FIG. 16 is a block diagram showing functions of an information processing device in a second embodiment.

FIG. 17 is a flowchart showing an example of a stressor action identification process in the second embodiment.

FIG. 18 is a diagram showing an information processing system in a third embodiment.

FIG. 19 is a sequence diagram showing an example of a process executed between the information processing device and an instrument management device in the third embodiment.

FIG. 20 is a block diagram showing functions of the information processing device in the third embodiment.

FIG. 21 is a diagram showing an example of an instrument information management table in the third embodiment.

FIG. 22 is a flowchart showing an example of a process executed by the information processing device in the third embodiment.

### MODE FOR CARRYING OUT THE INVENTION

Embodiments will be described below with reference to the drawings. The following embodiments are just examples and a variety of modifications are possible within the scope of the present disclosure.

### First Embodiment

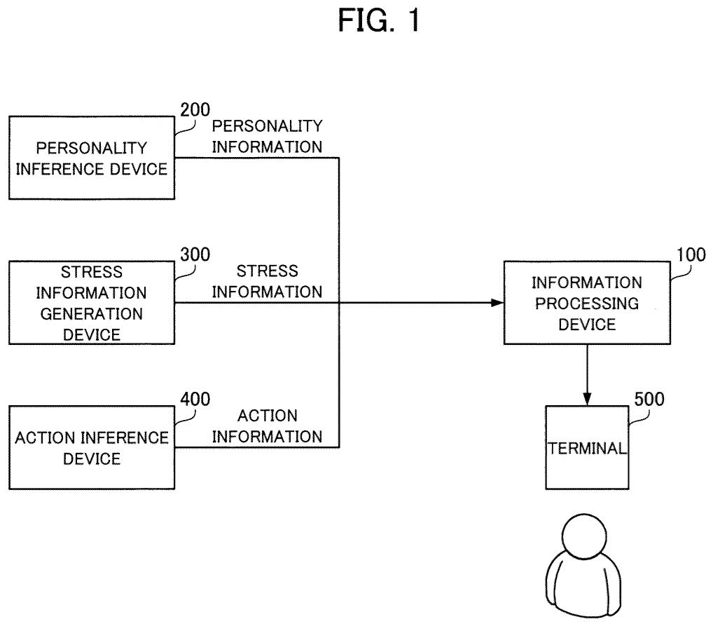

FIG. 1 is a diagram showing an information processing system in a first embodiment. The information processing system includes an information processing device 100, a personality inference device 200, a stress information generation device 300, an action inference device 400 and a terminal 500.

The information processing device 100, the personality inference device 200, the stress information generation device 300, the action inference device 400 and the terminal 500 execute communication via a network. The network is a wireless network, for example.

The information processing device 100 is a device that executes an identification method. The information processing device 100 is a server, for example.

The personality inference device 200 is a device that infers personality. The personality inference device 200 is a computer, for example.

The stress information generation device 300 is a device that generates stress information. The stress information is information regarding stress. The stress information is a stress value, for example. In the following description, the stress information is assumed to be the stress value. The stress value represents the degree of stress. The stress information generation device 300 is a computer, for example.

The action inference device 400 is a device that infers an action. The action inference device 400 is a computer, for example.

The terminal 500 is a device used by a user. The terminal 500 is a smartphone, a tablet terminal or a Personal Computer (PC), for example.

The information processing system will be described briefly below. The information processing device 100 acquires personality information from the personality inference device 200. The information processing device 100 acquires the stress information from the stress information generation device 300. The information processing device 100 acquires action information from the action inference device 400. The information processing device 100 identifies the stressor action by using the personality information, the stress information and the action information. The information processing device 100 transmits information indicating the stressor action to the terminal 500.

In the following, the information processing system will be described in detail.

First, hardware included in the information processing device 100 will be described below.

FIG. 2 is a diagram showing the hardware included in the information processing device in the first embodiment. The information processing device 100 includes a processor 101, a volatile storage device 102, a nonvolatile storage device 103 and an interface 104.

The processor 101 controls the whole of the information processing device 100. The processor 101 is a Central Processing Unit (CPU), a Field Programmable Gate Array (FPGA) or the like, for example. The processor 101 can also be a multiprocessor. Further, the information processing device 100 may include processing circuitry.

The volatile storage device 102 is main storage of the information processing device 100. The volatile storage device 102 is a Random Access Memory (RAM), for example. The nonvolatile storage device 103 is auxiliary storage of the information processing device 100. The nonvolatile storage device 103 is a Hard Disk Drive (HDD) or a Solid State Drive (SSD), for example.

The interface 104 executes communication with the personality inference device 200, the stress information generation device 300, the action inference device 400 and the terminal 500.

Further, each of the personality inference device 200, the stress information generation device 300 and the action inference device 400 includes a processor, a volatile storage device, a nonvolatile storage device and an interface similarly to the information processing device 100.

Next, hardware included in the terminal 500 will be described below.

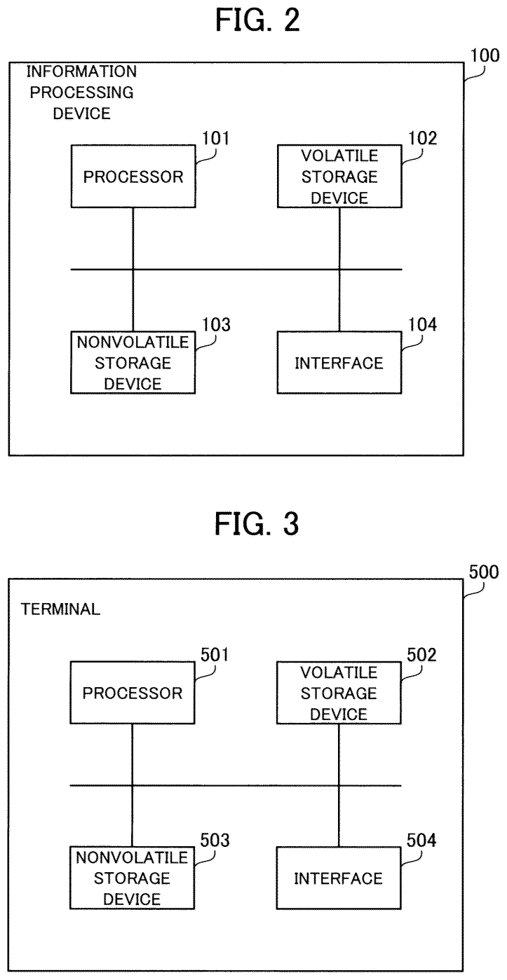

FIG. 3 is a diagram showing the hardware included in the terminal in the first embodiment. The terminal 500 includes a processor 501, a volatile storage device 502, a nonvolatile storage device 503 and an interface 504.

The processor 501 controls the whole of the terminal 500. The processor 501 is a CPU, an FPGA, a Digital Signal Processor (DSP) or the like, for example. The processor 501 can also be a multiprocessor. Further, the terminal 500 may include processing circuitry.

The volatile storage device 502 is main storage of the terminal 500. The volatile storage device 502 is a RAM, for example. The nonvolatile storage device 503 is auxiliary storage of the terminal 500. The nonvolatile storage device 503 is a HDD or an SSD, for example.

The interface 504 executes communication with the information processing device 100, the personality inference device 200, the stress information generation device 300 and the action inference device 400.

Next, a process executed between the information processing device 100 and the personality inference device 200 will be described below.

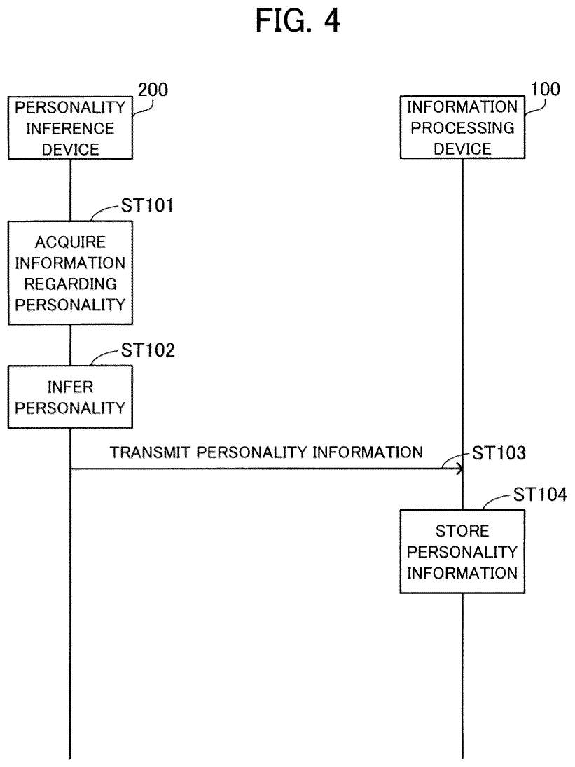

FIG. 4 is a sequence diagram showing an example of the process executed between the information processing device and the personality inference device in the first embodiment.

(Step ST101) The personality inference device 200 acquires information regarding personality of the user. For example, the personality inference device 200 acquires information inputted to the terminal 500 by the user (i.e., the information regarding the personality of the user) from the terminal 500. Incidentally, the information regarding the personality of the user may be referred to also as questionnaire information. It is also possible for the personality inference device 200 to acquire the information regarding the personality of the user from a device other than the terminal 500.

(Step ST102) The personality inference device 200 infers the personality of the user based on the information regarding the personality of the user. For example, the personality inference device 200 infers the personality of the user by using the information regarding the personality of the user and a learned model.

For example, the personality may be represented by big five personality traits. The big five personality traits are referred to also as a five-factor model. The big five personality traits are represented by openness, conscientiousness, extroversion, agreeableness and neuroticism. The openness indicates the degree of liking new experiences and diversity. The openness may be paraphrased as openness to experiences. The conscientiousness indicates the degree of a tendency to be aspiring and aiming for achievement or a tendency to like planned actions. The conscientiousness may be paraphrased as diligence. The extroversion indicates the degree of liking association or conversation with other people. The agreeableness indicates the degree of a tendency to be cooperative with other people. The agreeableness may be paraphrased as harmoniousness or attachment. The neuroticism indicates the degree of a tendency to be instable in personality and likely to experience unpleasant feeling. Further, the personality may also be represented by emotional stability instead of the neuroticism. Then, the personality is represented by parameters respectively representing the openness, the conscientiousness, the extroversion, the agreeableness and the neuroticism. The personality may also be represented by the degree of each of the openness, the conscientiousness, the extroversion, the agreeableness and the neuroticism. For example, the personality may be represented by high openness or low openness. Incidentally, the big five personality traits are described in Non-patent Reference 1.

Further, the personality may be represented by a parameter representing strength of self-control. The self-control means to pursue a desirable action and inhibit an undesirable action by a person's own will when the person faces temptation or impulse. Incidentally, the self-control is described in Non-patent Reference 2.

Furthermore, the personality may be represented by a parameter defined in the psychological field.

The personality inference device 200 may infer the personality of the user based on history records of inputting “like!” or the like to a Social Network Service (SNS) or history records of posting to an SNS. Incidentally, this inference is described in Non-patent Reference 3.

The personality inference device 200 may infer the personality of the user based on history records of operating the terminal 500, operation history records of the terminal 500, or information stored in the terminal 500. Incidentally, this inference is described in Non-patent Reference 4.

(Step ST103) The personality inference device 200 transmits the personality information indicating the inferred personality and a user identifier (ID) of the user to the information processing device 100. Here, the personality information can be represented as information indicating the personality of the user.

(Step ST104) The information processing device 100 stores the personality information while associating the user ID with the personality information.

Next, a process executed between the information processing device 100 and the stress information generation device 300 will be described below.

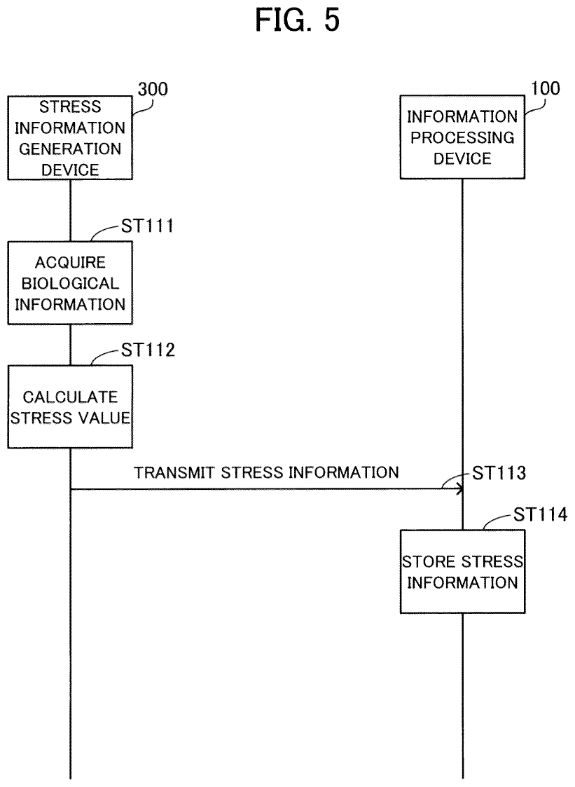

FIG. 5 is a sequence diagram showing an example of the process executed between the information processing device and the stress information generation device in the first embodiment.

(Step ST111) The stress information generation device 300 acquires information for generating information regarding the stress. For example, the stress information generation device 300 acquires information for calculating the stress value. Specifically, the stress information generation device 300 acquires the information for calculating the stress value from the terminal 500.

For example, the information for calculating the stress value is biological information. In the following description, the information for calculating the stress value is assumed to be the biological information. For example, the biological information is information based on a vital sign such as a heart rate, respiration or a pulse wave or physiological response such as brain waves, facial temperature, skin surface electric potential or eyeball movement. Further, for example, the biological information is information based on a component such as a hormone contained in blood, saliva or urine (e.g., amylase secretion volume in saliva). Furthermore, for example, the biological information is information based on a person's action such as facial expression or voice.

(Step ST112) The stress information generation device 300 calculates the stress value of the user based on the biological information. For example, when the biological information is the heart rate, the stress information generation device 300 calculates the stress value by performing frequency analysis on heart rate variability. Specifically, the stress information generation device 300 calculates a power spectrum from an interval (i.e., R-R interval) and a time between an R wave and an R wave in the heart rate variability in a constant period (e.g., 5 minutes). The stress information generation device 300 calculates the stress value by calculating a ratio between a frequency band (e.g., 0.004 Hz-0.15 Hz) and a high-frequency band (e.g., 0.15 Hz-0.4 Hz) of the power spectrum. The stress information generation device 300 may also calculate the stress value by using the average, the standard deviation or the like of the R-R interval.

Further, for example, when the biological information is the facial expression, the stress information generation device 300 infers emotion by using an image of the facial expression and an emotion inference model (i.e., learned model) and calculates the degree of the emotion such as anger or sorrow as the stress value.

(Step ST113) The stress information generation device 300 transmits the stress information, the user ID, and the date/time of the acquisition of the biological information to the information processing device 100. Incidentally, the date/time may be represented either by the time of day or a time length.

(Step ST114) The information processing device 100 stores the stress information while associating the user ID and the date/time with the stress information.

Next, a process executed between the information processing device 100 and the action inference device 400 will be described below.

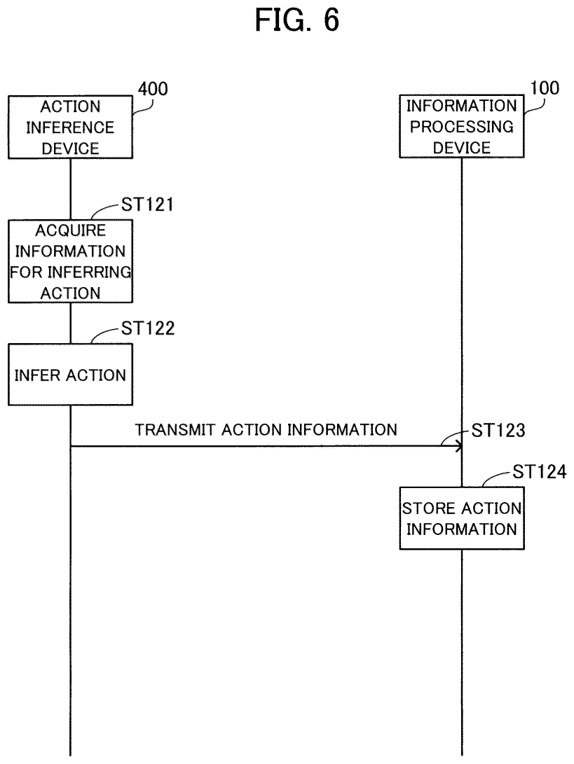

FIG. 6 is a sequence diagram showing an example of the process executed between the information processing device and the action inference device in the first embodiment.

(Step ST121) The action inference device 400 acquires information for inferring the action. For example, the action inference device 400 acquires the information for inferring the action from the terminal 500.

For example, the information for inferring the action is audio, a video or the like. Further, for example, the information for inferring the action is history records of operating an instrument, information acquired from a sensor, or the like. The information for inferring the action may include information indicating a date/time.

(Step ST122) The action inference device 400 infers the action based on the information for inferring the action. For example, the action inference device 400 infers the action by using the information for inferring the action and a learned model. The learned model can be a model based on a Long Short Term Memory (LSTM) network. Further, the learned model can be a model based on K-nearest neighbor algorithm, decision tree, random forest or Support Vector Machine (SVM).

For example, when the information for inferring the action indicates that an electric light has not been turned off for a certain period of time after being turned on, the action inference device 400 infers “forgetting to turn off an electric light”.

(Step ST123) The action inference device 400 transmits the action information indicating the inferred action, the user ID, and the date/time of the acquisition of the information for inferring the action to the information processing device 100. Incidentally, the date/time may be represented either by the time of day or a time length.

(Step ST124) The information processing device 100 stores the action information while associating the user ID and the date/time with the action information.

As above, the information processing device 100 receives the personality information, the stress information and the action information and stores the personality information, the stress information and the action information.

Next, functions of the information processing device 100 will be described below.

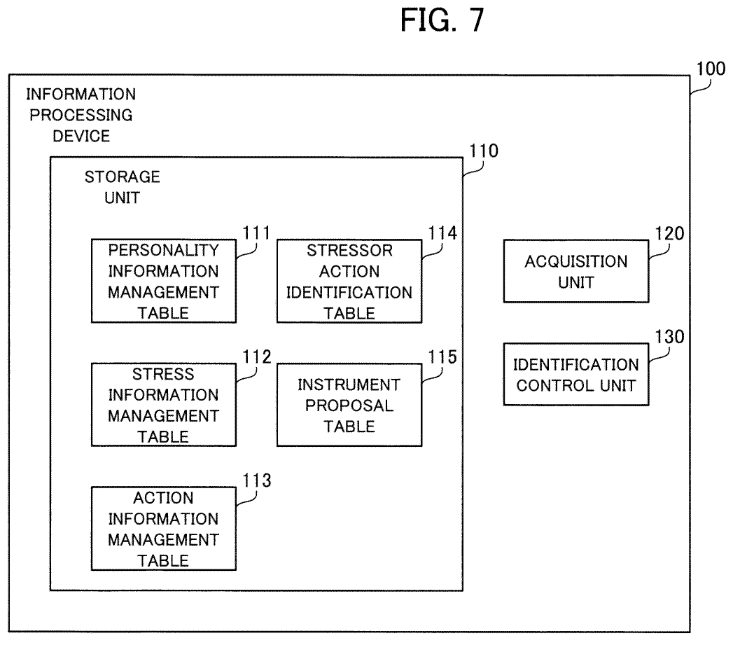

FIG. 7 is a block diagram showing the functions of the information processing device in the first embodiment. The information processing device 100 includes a storage unit 110, an acquisition unit 120 and an identification control unit 130.

The storage unit 110 may be implemented as a storage area reserved in the volatile storage device 102 or the nonvolatile storage device 103.

Part or all of the acquisition unit 120 and the identification control unit 130 may be implemented by processing circuitry. Further, part or all of the acquisition unit 120 and the identification control unit 130 may be implemented as modules of a program executed by the processor 101. For example, the program executed by the processor 101 is referred to also as an identification program. The identification program has been recorded in a record medium, for example.

The storage unit 110 stores a personality information management table 111. When the personality information is acquired, the information processing device 100 registers the personality information in the personality information management table 111. An example of the personality information management table 111 will be shown below.

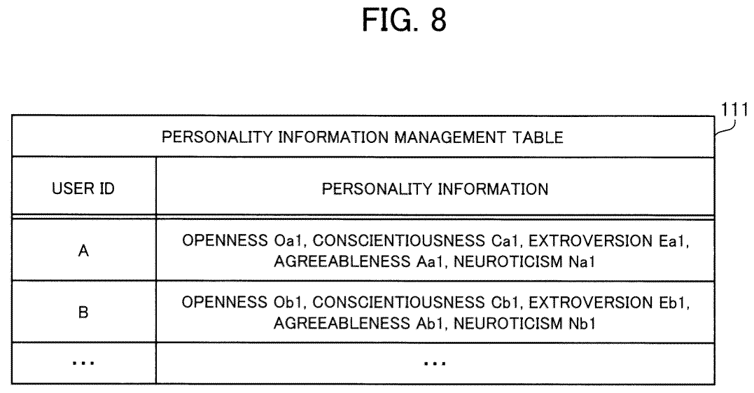

FIG. 8 is a diagram showing an example of the personality information management table in the first embodiment. The personality information management table 111 includes items of user ID and personality information.

For example, FIG. 8 indicates that the personality information corresponding to a user ID “A” is openness “Oa1”, conscientiousness “Ca1”, extroversion “Ea1”, agreeableness “Aa1”, and neuroticism “Na1”.

Further, for example, FIG. 8 indicates that the personality information corresponding to a user ID “B” is openness “Ob1”, conscientiousness “Cb1”, extroversion “Eb1”, agreeableness “Ab1”, and neuroticism “Nb1”.

The storage unit 110 stores a stress information management table 112. When the stress information is acquired, the information processing device 100 registers the stress information in the stress information management table 112. An example of the stress information management table 112 will be shown below.

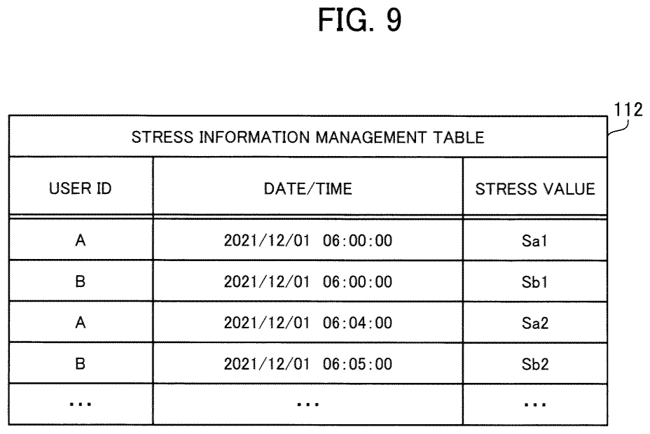

FIG. 9 is a diagram showing an example of the stress information management table in the first embodiment. The stress information management table 112 includes items of user ID, date/time and stress value.

In the stress information management table 112, a fact that a date/time of the acquisition of the biological information is “2021 Dec. 1 06:00:00” has been registered. Further, FIG. 9 indicates that the stress value corresponding to the user ID “A” is “Sal”.

Furthermore, in the stress information management table 112, a fact that a date/time of the acquisition of the biological information is “2021 Dec. 1 06:00:00” has been registered. Moreover, FIG. 9 indicates that the stress value corresponding to the user ID “B” is “Sb1”.

The storage unit 110 stores an action information management table 113. When the action information is acquired, the information processing device 100 registers the action information in the action information management table 113. An example of the action information management table 113 will be shown below.

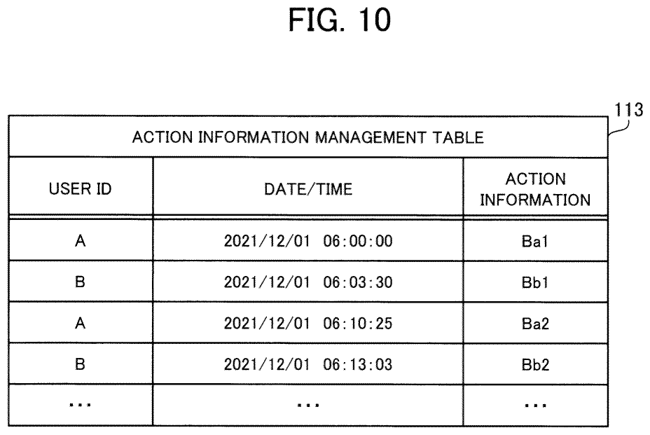

FIG. 10 is a diagram showing an example of the action information management table in the first embodiment. The action information management table 113 includes items of user ID, date/time and action information.

In the action information management table 113, a fact that a date/time of the acquisition of information for identifying the action is “2021 Dec. 1 06:00:00” has been registered. Further, FIG. 10 indicates that the action information corresponding to the user ID “A” is “Ba1”.

In the action information management table 113, a fact that a date/time of the acquisition of information for identifying the action is “2021 Dec. 1 06:03:30” has been registered. Further, FIG. 10 indicates that the action information corresponding to the user ID “B” is “Bb1”.

The storage unit 110 may store a stressor action identification table 114. An example of the stressor action identification table 114 will be shown below.

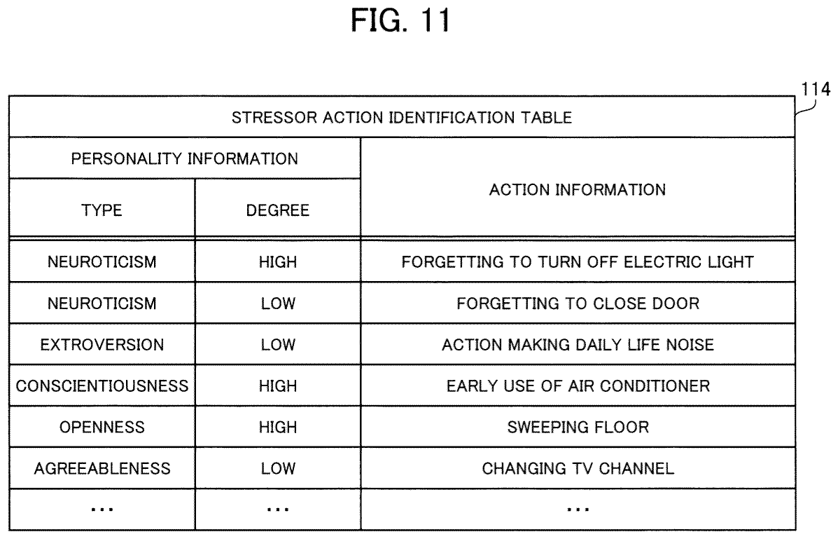

FIG. 11 is a diagram showing an example of the stressor action identification table in the first embodiment. The stressor action identification table 114 is referred to also as stressor action identification information. The stressor action identification table 114 is information indicating a correspondence relationship between the personality information and the action information. For example, the personality information in the stressor action identification table 114 includes items of type and degree.

The stressor action identification table 114 is used for identifying the stressor action. For example, when the personality information indicates high neuroticism and the action information indicates the “forgetting to turn off an electric light”, a record in which the type is “neuroticism”, the degree is “high”, and the action information is the “forgetting to turn off an electric light” is referred to. Then, the “forgetting to turn off an electric light” is identified as the stressor action.

When the type of the personality information is represented by a parameter, the following handling is carried out. For example, when the parameter of the neuroticism is greater than or equal to a threshold value, the neuroticism is treated as high. Further, for example, when the parameter of the neuroticism is less than the threshold value, the neuroticism is treated as low. As above, when the type of the personality information is represented by a parameter, the stressor action is identified by using the threshold value.

As another method, it is also possible to identify the stressor action by aggregating results in which each type of the personality information is represented by one of two values: “presence” and “absence”. Further, it is also possible to identify the stressor action based on a result of performing statistical processing on distribution data obtained by plotting the parameters of the types in a region represented by a plurality of dimensions.

The storage unit 110 may store an instrument proposal table 115. An example of the instrument proposal table 115 will be shown below.

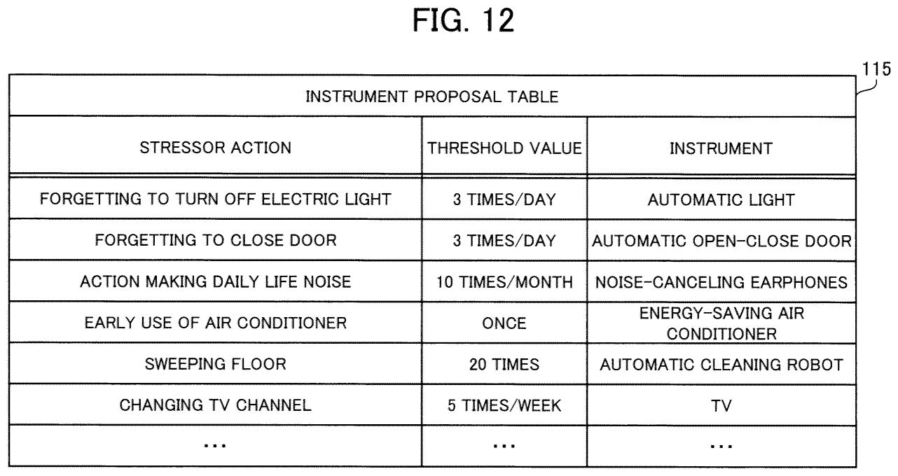

FIG. 12 is a diagram showing an example of the instrument proposal table in the first embodiment. The instrument proposal table 115 is referred to also as instrument proposal information. The instrument proposal table 115 is information indicating an instrument to be proposed when the stressor action has been performed greater than or equal to a threshold value. Specifically, the instrument proposal table 115 includes items of stressor action, threshold value and instrument. In the item of instrument, an instrument as an object of the proposal is registered.

The acquisition unit 120 acquires the personality information, the stress information and the action information from the storage unit 110. It is also possible for the acquisition unit 120 to acquire the personality information, the stress information and the action information not stored in the storage unit 110 from an external device. The external device is a device capable of connecting to the information processing device 100. Illustration of the external device is left out.

The acquisition unit 120 acquires the stressor action identification table 114. For example, the acquisition unit 120 acquires the stressor action identification table 114 from the storage unit 110. For example, the acquisition unit 120 acquires the stressor action identification table 114 from an external device.

Further, the acquisition unit 120 acquires the instrument proposal table 115. For example, the acquisition unit 120 acquires the instrument proposal table 115 from the storage unit 110 or an external device.

Functions of the identification control unit 130 will be described later in detail.

Next, a process executed by the information processing device 100 will be described below by using a flowchart.

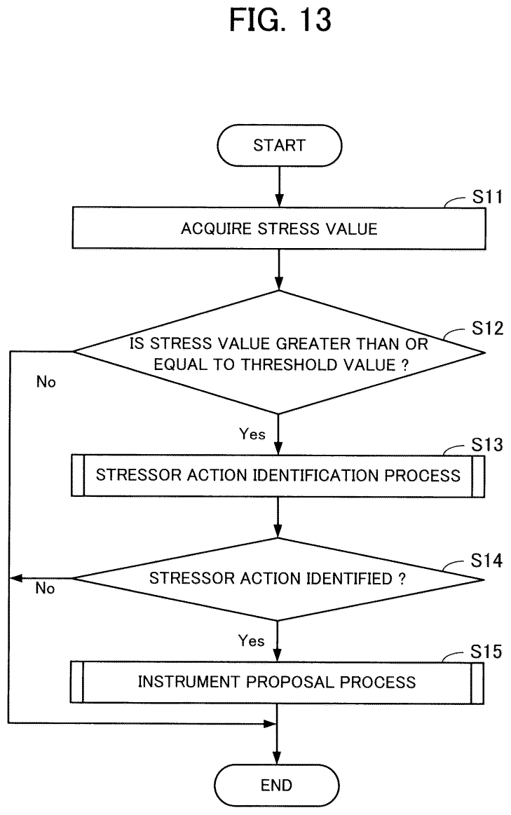

FIG. 13 is a flowchart showing an example of the process executed by the information processing device in the first embodiment. The following description will be given of a case where the stressor action is identified by using the user ID “A”. The user with the user ID “A” is referred to also as a first user.

(Step S11) The acquisition unit 120 acquires the stress value regarding the user ID “A” in the stress information management table 112.

(Step S12) The identification control unit 130 judges whether or not the stress value is greater than or equal to a predetermined threshold value. If the stress value is greater than or equal to the threshold value, the process advances to step S13. If the stress value is less than the threshold value, the process ends.

(Step S13) The identification control unit 130 executes a stressor action identification process.

(Step S14) The identification control unit 130 judges whether or not the stressor action has been identified. If the stressor action has been identified, the process advances to step S15. If the stressor action has not been identified, the process ends.

(Step S15) The identification control unit 130 executes an instrument proposal process.

In the step S12, a case of using the stress value as the stress information has described. The stress information can also be information indicating whether there is stress or not. When the stress information is such information, the identification control unit 130 in the step S12 judges whether there is stress or not. If there is stress, the process advances to the step S13. If there is no stress, the process ends. Further, the stress information can also be a stress value represented by a plurality of elements such as emotion and a fatigue level. When the stress information is such a stress value, the identification control unit 130 in the step S12 judges whether or not the stress value is included in a specific element region. Incidentally, the specific element region is, for example, a region represented by a plurality of dimensions for making a statistical judgment by plotting the aforementioned emotion, the fatigue level, the parameters of the types of the personality information, and the like. That is, the specific element region is a region for judging whether there is stress or not. If the stress value is included in the specific element region, the process advances to the step S13. If the stress value is not included in the specific element region, the process ends. It is also possible to implement the step S12 and the step S13 by one processing step.

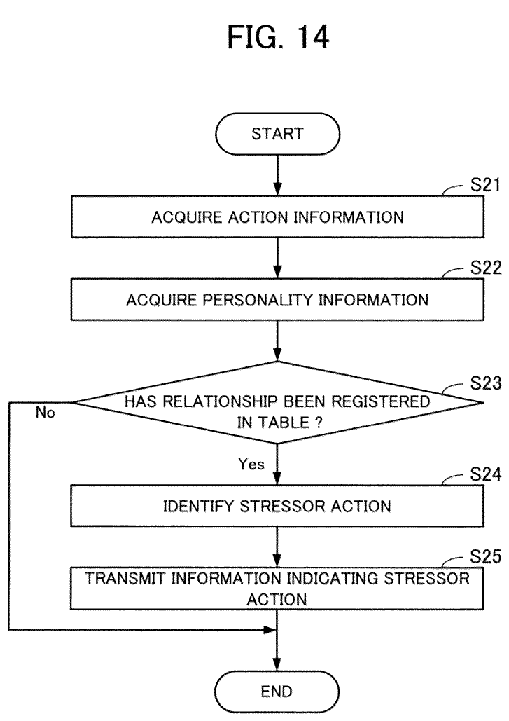

FIG. 14 is a flowchart showing an example of the stressor action identification process in the first embodiment. The process of FIG. 14 corresponds to the processing in the step S13.

(Step S21) The acquisition unit 120 acquires the action information regarding a user ID other than the user ID “A” that is related with the stress value acquired in the step S11. In detail, the identification control unit 130 acquires the action information regarding a user ID other than the user ID “A” based on a date/time corresponding to the stress value. Specifically, the acquisition unit 120 acquires the action information regarding a user ID other than the user ID “A” that is with a date/time the same as the date/time corresponding to the stress value or with a date/time before the date/time corresponding to the stress value. For example, the date/time corresponding to the stress value is assumed to be “2021 Dec. 1 06:04:00”. The acquisition unit 120 acquires the action information “Bb1” regarding the user ID “B” that is with the date/time “2021 Dec. 1 06:03:30”.

Further, a user with the user ID other than the user ID “A” is referred to also as a second user.

(Step S22) The acquisition unit 120 acquires the personality information regarding the user ID “A”.

(Step S23) The identification control unit 130 judges whether or not a relationship between the personality information and the action information has been registered in the stressor action identification table 114.

For example, the personality information is assumed to indicate “high neuroticism”. Further, the action information is assumed to indicate the “forgetting to turn off an electric light”. The identification control unit 130 judges that the relationship between the personality information and the action information has been registered in the stressor action identification table 114.

If the condition is satisfied, the process advances to step S24. If the condition is not satisfied, the process ends.

(Step S24) The identification control unit 130 identifies the action information as the stressor action. For example, the identification control unit 130 identifies the “forgetting to turn off an electric light” as the stressor action.

(Step S25) The identification control unit 130 transmits information indicating the stressor action to the terminal 500.

The terminal 500 displays the stressor action. This enables the user to recognize the stressor action.

Further, the identification control unit 130 may transmit the information indicating the stressor action to a terminal of a person (e.g., the user with the user ID “B”) living together with the user (e.g., the user with the user ID “A”). Furthermore, the transmission of the information indicating the stressor action may also be performed by means of unicast, broadcast or multicast.

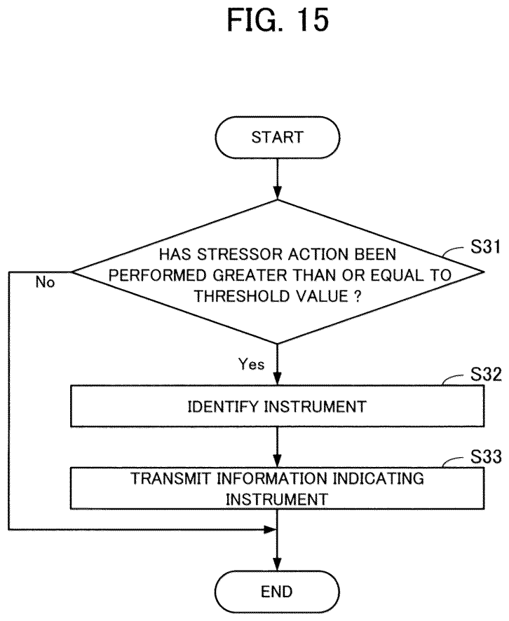

FIG. 15 is a flowchart showing an example of the instrument proposal process in the first embodiment. The process of FIG. 15 corresponds to the processing in the step S15.

(Step S31) The identification control unit 130 judges whether the identified stressor action has been performed greater than or equal to the threshold value or not, by using the instrument proposal table 115. If the stressor action has been performed greater than or equal to the threshold value, the process advances to step S32. If the stressor action has not been performed greater than or equal to the threshold value, the identification control unit 130 adds 1 to an action count corresponding to the stressor action. Then, the process ends.

(Step S32) The identification control unit 130 identifies an instrument corresponding to the stressor action based on the instrument proposal table 115. For example, when the action of “forgetting to turn off an electric light” has been performed three times or more in one day (i.e., when the action count of the “forgetting to turn off an electric light” reaches 3 or more in one day), the identification control unit 130 identifies an automatic light.

(Step S33) The identification control unit 130 transmits information indicating the identified instrument to the terminal 500. The transmission of this information means proposal of purchase or rental of the identified instrument.

The terminal 500 displays the instrument. This enables the user to know a method for eliminating the stressor action.

Further, the identification control unit 130 may transmit the information indicating the identified instrument to a terminal of a person (e.g., the user with the user ID “B”) living together with the user (e.g., the user with the user ID “A”). Furthermore, the transmission of the information indicating the identified instrument may also be performed by means of unicast, broadcast or multicast.

In FIG. 15, the case where an instrument is proposed based on the number of times (count) of the stressor action has described. It is also possible for the information processing device 100 to immediately propose an instrument when the stress is high. A process executed by the information processing device 100 will be described below. First, the threshold value regarding the stress value is registered in the item “threshold value” of the instrument proposal table 115. For example, in the instrument proposal table 115, a threshold value “X1” regarding the stress value corresponding to the stressor action “forgetting to turn off an electric light” is registered. In the step S31, the identification control unit 130 judges whether or not the stress value acquired in the step S11 is greater than or equal to the threshold value corresponding to the identified stressor action. If the stress value is greater than or equal to the threshold value, the process advances to the step S32. If the stress value is less than the threshold value, the process ends. For example, when the identified stressor action is the “forgetting to turn off an electric light”, the information processing device 100 proposes the “automatic light” if the stress value acquired in the step S11 is greater than or equal to “X1”.

Further, the above description has been given of the case where the stress information is the stress value. The stress information can also be information indicating whether there is stress or not. When the stress information is such information, the identification control unit 130 in the step S31 judges whether there is stress or not. If there is stress, the process advances to the step S32. If there is no stress, the process ends. Further, the stress information can also be a stress value represented by a plurality of elements such as the emotion and the fatigue level. When the stress information is such a stress value, the identification control unit 130 in the step S31 judges whether or not the stress value is included in a specific element region. Incidentally, the specific element region is, for example, a region represented by a plurality of dimensions for making a statistical judgment by plotting the aforementioned emotion, the fatigue level, the parameters of the types of the personality information, and the like. That is, the specific element region is a region for judging whether there is stress or not. If the stress value is included in the specific element region, the process advances to the step S32. If the stress value is not included in the specific element region, the process ends.

As above, the identification control unit 130 judges whether an instrument corresponding to the stressor action should be proposed or not based on the stress information (step S31). If the proposal should be made (Yes in the step S31), the identification control unit 130 identifies an instrument corresponding to the identified stressor action based on the instrument proposal table 115 (step S32). The identification control unit 130 transmits the information indicating the identified instrument (step S33).

According to the first embodiment, the stressor action is not identified by use of the action information as in the Patent Reference 1, for example. The information processing device 100 identifies the stressor action by use of the personality information, the stress information and the action information. Accordingly, the stressor action is identified with high accuracy. Thus, the information processing device 100 is capable of increasing the identification accuracy of the stressor action.

### Second Embodiment

Next, a second embodiment will be described below. In the second embodiment, the description will be given mainly of features different from those in the first embodiment. In the second embodiment, the description is omitted for features in common with the first embodiment.

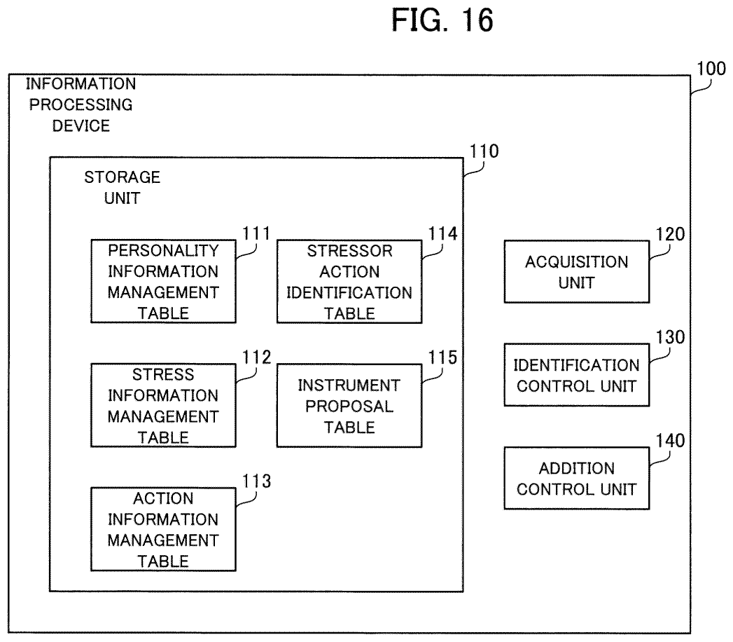

FIG. 16 is a block diagram showing functions of an information processing device in the second embodiment. Each component in FIG. 16 that is the same as a component shown in FIG. 7 is assigned the same reference character as in FIG. 7.

The information processing device 100 further includes an addition control unit 140. Part or the whole of the addition control unit 140 may be implemented by processing circuitry. Further, part or the whole of the addition control unit 140 may be implemented as modules of a program executed by the processor 101.

Functions of the addition control unit 140 will be described later in detail.

Next, a process executed by the information processing device 100 will be described below by using a flowchart.

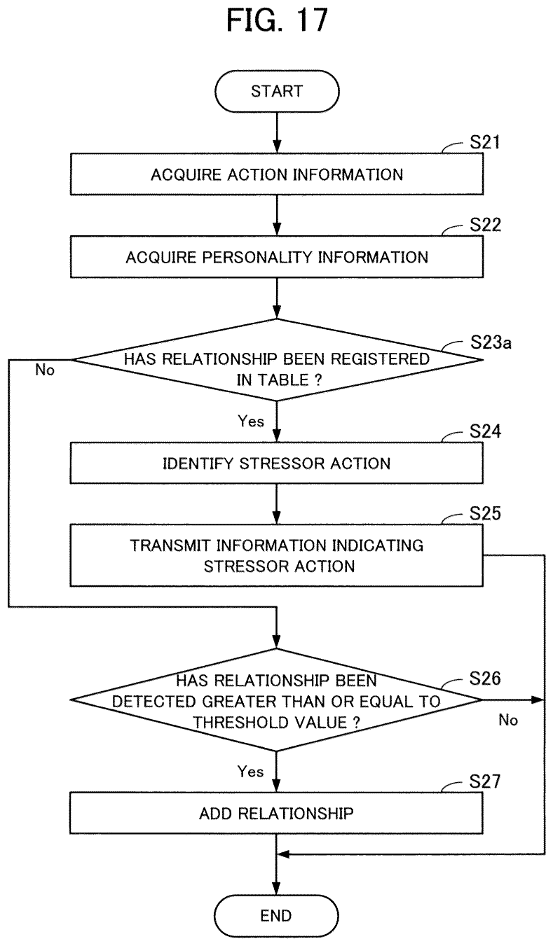

FIG. 17 is a flowchart showing an example of a stressor action identification process in the second embodiment. The process of FIG. 17 differs from the process of FIG. 14 in that steps S23a, S26 and S27 are executed. Thus, the steps S23a, S26 and S27 in FIG. 17 will be described below. Then, the description will be omitted for processing other than the steps S23a, S26 and S27.

(Step S23a) The identification control unit 130 judges whether or not a relationship between the personality information and the action information has been registered in the stressor action identification table 114. If the condition is satisfied, the process advances to the step S24. If the condition is not satisfied, the process advances to the step S26.

(Step S26) The addition control unit 140 judges whether or not a relationship between the personality information and the action information has been detected greater than or equal to a predetermined threshold value. If the condition is satisfied, the process advances to the step S27. If the condition is not satisfied, the process ends.

(Step S27) The addition control unit 140 adds the relationship between the personality information and the action information to the stressor action identification table 114.

According to the second embodiment, the information processing device 100 registers a relationship between a newly occurring action and personality in the stressor action identification table 114. Accordingly, the information processing device 100 is capable of identifying a newly occurring action as the stressor action.

### Third Embodiment

Next, a third embodiment will be described below. In the third embodiment, the description will be given mainly of features different from those in the second embodiment. In the third embodiment, the description is omitted for features in common with the second embodiment.

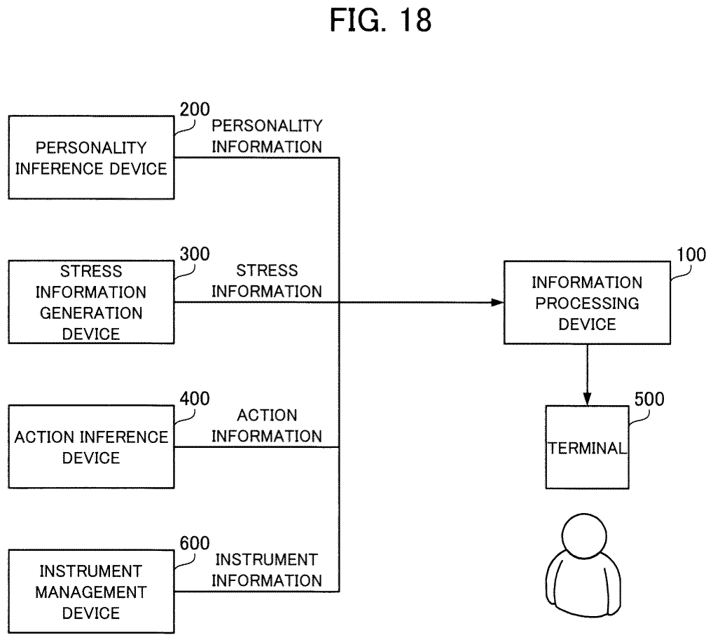

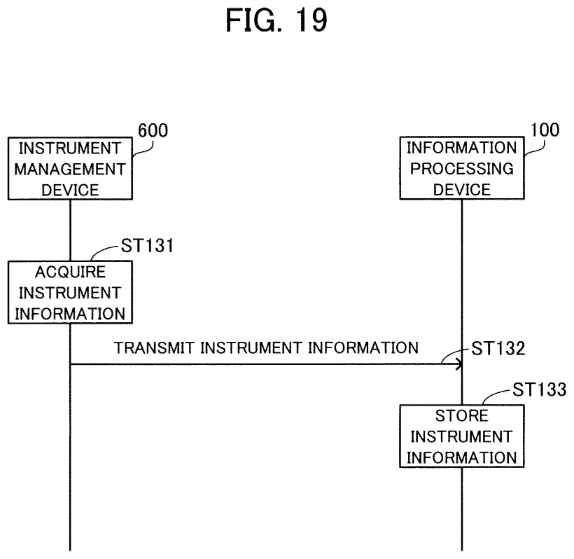

FIG. 18 is a diagram showing an information processing system in the third embodiment. The information processing system further includes an instrument management device 600. The information processing device 100 and the instrument management device 600 execute communication via a network. The instrument management device 600 is a computer, for example.

Next, a process executed between the information processing device 100 and the instrument management device 600 will be described below.

FIG. 19 is a sequence diagram showing an example of the process executed between the information processing device and the instrument management device in the third embodiment.

(Step ST131) The instrument management device 600 acquires instrument information indicating an instrument. For example, the instrument is a newly purchased instrument. Further, for example, the instrument is a newly rented instrument.

(Step ST132) The instrument management device 600 transmits the instrument information and the date/time of the acquisition of the instrument information to the information processing device 100. Incidentally, the date/time may be represented either by the time of day or a time length.

(Step ST133) The information processing device 100 stores the instrument information while associating the date/time with the instrument information.

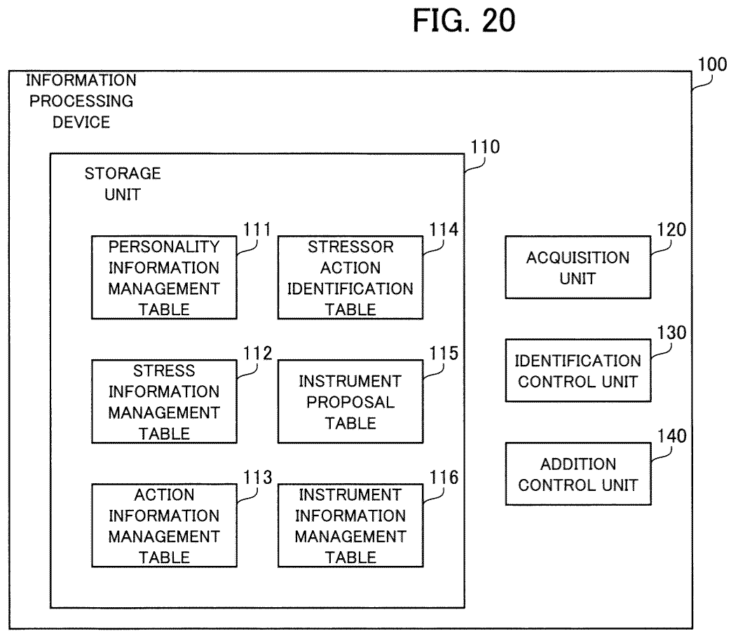

FIG. 20 is a block diagram showing functions of the information processing device in the third embodiment. Each component in FIG. 20 that is the same as a component shown in FIG. 16 is assigned the same reference character as in FIG. 16.

The storage unit 110 stores an instrument information management table 116. When the instrument information is acquired, the information processing device 100 registers the instrument information in the instrument information management table 116. An example of the instrument information management table 116 will be shown below.

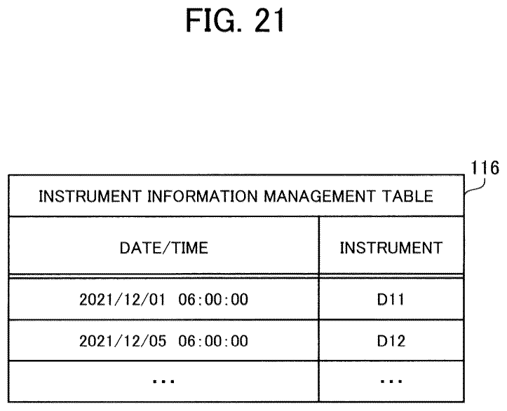

FIG. 21 is a diagram showing an example of the instrument information management table in the third embodiment. The instrument information management table 116 includes items of date/time and instrument.

In the instrument information management table 116, a fact that a date/time of the acquisition of the instrument information is “2021 Dec. 1 06:00:00” has been registered. Further, FIG. 21 indicates that an instrument “D11” has been registered in the instrument information management table 116.

Furthermore, in the instrument information management table 116, a fact that a date/time of the acquisition of the instrument information is “2021 Dec. 5 06:00:00” has been registered. Moreover, FIG. 21 indicates that an instrument “D12” has been registered in the instrument information management table 116.

The acquisition unit 120 acquires the instrument information from the storage unit 110. It is also possible for the acquisition unit 120 to acquire the instrument information not stored in the storage unit 110 from an external device.

Functions of the addition control unit 140 will be described later in detail.

Next, a process executed by the information processing device 100 will be described below by using a flowchart.

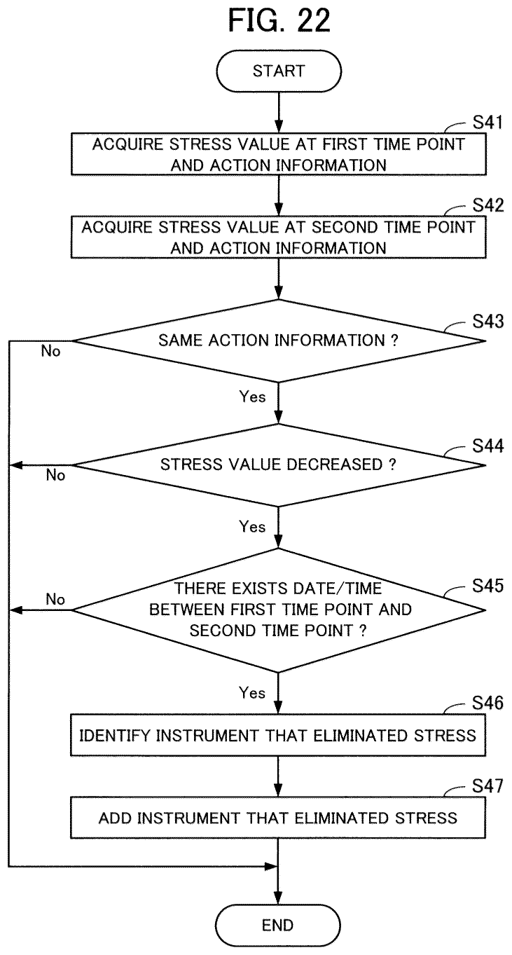

FIG. 22 is a flowchart showing an example of the process executed by the information processing device in the third embodiment.

(Step S41) The acquisition unit 120 acquires the stress value regarding the user ID “A” at a first time point (hereinafter referred to as a first stress value). The acquisition unit 120 acquires the action information (hereinafter referred to as first action information) regarding a user ID other than the user ID “A” that is related with the first stress value. In the following description, the user ID other than the user ID “A” is assumed to be the user ID “B”.

For example, a date/time corresponding to the stress value regarding the user ID “A” at the first time point is assumed to be “2021 Dec. 1 06:04:00”. The acquisition unit 120 acquires the action information regarding the user ID “B” at “2021 Dec. 1 06:03:30”.

Incidentally, the acquisition unit 120 acquires the first stress value and the first action information from the storage unit 110. It is also possible for the acquisition unit 120 to acquire the first stress value and the first action information from an external device.

(Step S42) The acquisition unit 120 acquires the stress value regarding the user ID “A” at a second time point (hereinafter referred to as a second stress value). The acquisition unit 120 acquires the action information (hereinafter referred to as second action information) regarding the user ID “B” that is related with the second stress value.

Here, the second time point is a time point after the first time point.

For example, a date/time corresponding to the stress value regarding the user ID “A” at the second time point is assumed to be “2021 Dec. 10 06:04:00”. The acquisition unit 120 acquires the action information regarding the user ID “B” at “2021 Dec. 10 06:03:30”.

Incidentally, the acquisition unit 120 acquires the second stress value and the second action information from the storage unit 110. It is also possible for the acquisition unit 120 to acquire the second stress value and the second action information from an external device.

(Step S43) The addition control unit 140 judges whether or not the first action information and the second action information are the same as each other. If the first action information and the second action information are the same as each other, the process advances to step S44. If the first action information and the second action information differ from each other, the process ends.

(Step S44) The addition control unit 140 judges whether or not the second stress value is less than the first stress value. If the second stress value is less than the first stress value, the process advances to step S45. If the second stress value is greater than or equal to the first stress value, the process ends.

(Step S45) The addition control unit 140 judges whether or not there exists a date/time between the first time point and the second time point by referring to the instrument information management table 116. If such a date/time exists, the process advances to step S46. If such a date/time does not exist, the process ends.

(Step S46) The addition control unit 140 judges that an instrument corresponding to the date/time is an instrument that eliminated the stress.

For example, the addition control unit 140 judges that there exists “2021 Dec. 5 06:00:00” as the date/time (referred to also as a third time point) between the first time point and the second time point (Yes in the step S45). The addition control unit 140 judges that the instrument “D12” corresponding to “2021 Dec. 5 06:00:00” is the instrument that eliminated the stress.

Incidentally, the instrument information indicating the instrument corresponding to the third time point is acquired by the acquisition unit 120. Specifically, the acquisition unit 120 acquires the instrument information from the storage unit 110. It is also possible for the acquisition unit 120 to acquire the instrument information from an external device.

(Step S47) The addition control unit 140 adds a relationship between the first action information or the second action information and the instrument that eliminated the stress to the instrument proposal table 115. Further, a threshold value corresponding to the first action information or the second action information and the instrument that eliminated the stress may be set arbitrarily.

According to the third embodiment, the information processing device 100 adds an instrument that eliminates stress. This addition makes it possible for the information processing device 100 to propose the instrument.

Features in the embodiments described above can be appropriately combined with each other.

### DESCRIPTION OF REFERENCE CHARACTERS

100: information processing device, 101: processor, 102: volatile storage device, 103: nonvolatile storage device, 104: interface, 110: storage unit, 111: personality information management table, 112: stress information management table, 113: action information management table, 114: stressor action identification table, 115: instrument proposal table, 116: instrument information management table, 120: acquisition unit, 130: identification control unit, 140: addition control unit, 200: personality inference device, 300: stress information generation device, 400: action inference device, 500: terminal, 501: processor, 502: volatile storage device, 503: nonvolatile storage device, 504: interface, 600: instrument management device

1. An information processing device comprising:
acquiring circuitry to acquire stressor action identification information indicating a correspondence relationship between personality information and action information, personality information indicating personality of a first user, stress information as information regarding stress on the first user, and action information indicating an action of a second user; and
identification controlling circuitry to judge whether a relationship between the personality information and the action information has been registered in the stressor action identification information or not based on the stress information and identify the action information as a stressor action if the relationship has been registered in the stressor action identification information.

2. The information processing device according to claim 1, wherein the identification controlling circuitry transmits information indicating the identified stressor action.

3. The information processing device according to claim 1, further comprising addition controlling circuitry to judge whether the relationship has been detected greater than or equal to a predetermined threshold value or not if the relationship has not been registered in the stressor action identification information and add the relationship to the stressor action identification information if the relationship has been detected greater than or equal to the threshold value.

4. The information processing device according to claim 1, wherein
the acquiring circuitry acquires instrument proposal information indicating an instrument to be proposed when a stressor action has been performed greater than or equal to a threshold value, and
the controlling circuitry judges whether the identified stressor action has been performed greater than or equal to a threshold value or not by using the instrument proposal information, and if the identified stressor action has been performed greater than or equal to the threshold value, identifies an instrument corresponding to the identified stressor action based on the instrument proposal information and transmits information indicating the identified instrument.

5. The information processing device according to claim 1, wherein
the acquiring circuitry acquires instrument proposal information indicating an instrument corresponding to a stressor action, and
the identification controlling circuitry judges whether an instrument corresponding to the identified stressor action should be proposed or not based on the stress information, and if the proposal should be made, identifies an instrument corresponding to the identified stressor action based on the instrument proposal information and transmits information indicating the identified instrument.

6. The information processing device according to claim 4, further comprising addition controlling circuitry, wherein
the acquiring circuitry acquires a first stress value as a degree of stress on the first user at a first time point, first action information as action information regarding the second user that is related with the first stress value, a second stress value as the degree of stress on the first user at a second time point as a time point after the first time point, second action information as action information regarding the second user that is related with the second stress value, and instrument information indicating an instrument corresponding to a third time point as a time point between the first time point and the second time point, and
when the first action information and the second action information are a same as each other, the addition controlling circuitry judges whether or not the second stress value is less than the first stress value, and if the second stress value is less than the first stress value, the addition controlling circuitry adds a relationship between the first action information or the second action information and the instrument corresponding to the third time point to the instrument proposal information.

7. An information processing system comprising:
a personality inference device that acquires information regarding personality of a first user, infers the personality of the first user based on the information, and transmits personality information indicating the personality of the first user;
a stress information generation device that acquires information for generating information regarding stress on the first user, generates stress information based on the information, and transmits the stress information;
an action inference device that acquires information for inferring an action of a second user, infers the action of the second user based on the information, and transmits action information indicating the action of the second user; and
an information processing device,
wherein the information processing device includes:
acquiring circuitry to acquire stressor action identification information indicating a correspondence relationship between personality information and action information, the personality information transmitted by the personality inference device, the stress information transmitted by the stress information generation device, and the action information transmitted by the action inference device; and
identification controlling circuitry to judge whether a relationship between the personality information and the action information has been registered in the stressor action identification information or not based on the stress information and identify the action information as a stressor action if the relationship has been registered in the stressor action identification information.

8. An identification method performed by an information processing device, the identification method comprising:
acquiring stressor action identification information indicating a correspondence relationship between personality information and action information, personality information indicating personality of a first user, stress information as information regarding stress on the first user, and action information indicating an action of a second user;
judging whether a relationship between the personality information and the action information has been registered in the stressor action identification information or not based on the stress information; and
identifying the action information as a stressor action if the relationship has been registered in the stressor action identification information.

9. An information processing device comprising:
a processor to execute a program; and
a memory to store the program which, when executed by the processor, performs processes of,
acquiring stressor action identification information indicating a correspondence relationship between personality information and action information, personality information indicating personality of a first user, stress information as information regarding stress on the first user, and action information indicating an action of a second user,
judging whether a relationship between the personality information and the action information has been registered in the stressor action identification information or not based on the stress information, and
identifying the action information as a stressor action if the relationship has been registered in the stressor action identification information.
